(19) United States
(12) Patent Application Publication
(10) Pub. No.: US 2007/0239268 A1
(43) Pub. Date: Oct. 11, 2007

(54) Adhesion resistant implantable device

(76) Inventors: Lee Fox, Jupiter, FL (US); Peter Haaland, Louisville, CO (US); Michael Kalter, Tequesta, FL (US)

Correspondence Address: MCHALE & SLAVIN, P.A., 2855 PGA BLVD, PALM BEACH GARDENS, FL, 33410, US

(21) Appl. No.: 11/394,141

(22) Filed: Mar. 30, 2006

(51) Int. Cl. A61F 2/06

(52) U.S. Cl. 623/1.46

(57) ABSTRACT

The present invention discloses implantable devices that resist adhesion of colloidal particles such as are present in biological fluids, and methods for their manufacture. In a particular embodiment, the device may be an endovascular stent and a method for its production, for reducing, and preferably eliminating, restenosis. This objective is accomplished by recognizing the fundamental coupling between the surface texture and composition, on one hand, and the drag and adhesive forces acting on a colloidal particle, on the other. The surfaces of the device are first exposed to fluid flow whereby they are polished via a micro and/or nano-abrasive media so that they are featureless on length scales that are commensurate with the sizes of colloidal particles that initiate restenosis. Secondly, the surface is treated with a thin coating that reduces, or preferably eliminates, hydrogen bonding with colloidal particles. In one embodiment, processes for treatment of such implantable devices are taught which result in targeted reduction of structural micro-anomalies in such devices and targeted reduction or elimination of the propensity for occlusive deposits to form therein, whereby properties of selective adherence of particular cell types are derived.

### FIELD OF THE INVENTION

This invention relates to implantable devices that resist adhesion of colloidal material when immersed in biological fluids, and methods for their manufacture; particularly to adhesion resistant implantable stents, and processes for their manufacture.

### BACKGROUND OF THE INVENTION

Circulation of fluid within living organisms is vitally important and embraces transport of colloidal suspensions such as blood, urine, lymph, and the like throughout the body. Various benign and malignant conditions can cause obstruction of these flows, which in the past required highly invasive surgical interventions.

Since its inception in the late 1970s balloon angioplasty has become increasingly popular as a less invasive method for revascularization of coronary patients with diseased arteries. This has led to the development of new percutaneous devices to treat atherosclerotic vasculopathies. However, the expanded use of angioplasty has shown that the arteries, as well as other vessels, react to angioplasty by a proliferative process similar to wound healing that limits the success of the treatment modality. This process is known as restenosis. Restenosis is defined as a re-narrowing of the treated segment, which reduces the lumen diameter to less than half that of the adjacent normal segment of the vessel in the adjacent normal segment of the artery. Depending on the patient population studied, the restenosis rates range from 30% to 44% of lesions treated by balloon dilation.

The pervasiveness of this problem has led practitioners to develop various endovascular techniques to minimize the risk of restenosis; and caused such practitioners to gauge the ultimate efficacy and measure of success of any interventional method by not only how quickly or dependably it opens the diseased artery, but also how likely it is to trigger restenosis.

Several interventional devices and procedures have been introduced with the aim of reducing the immediate and short term restenosis rate of balloon angioplasty. Two of the most utilized devices/techniques are: 1) atherectomy, or tissue removing techniques; and 2) stenting, or vascular splinting techniques, which involve implantation of a rigid structure within a vessel to restore fluid flow. The ways in which these techniques open vessels differ substantively, as do the manner in which they promulgate restenosis.

There is a significant reduction in restenosis rates with placement of an endovascular stent. The purpose of such stenting is to maintain the vessel lumen by providing intraluminal radial support. Stents can be made of a variety of metals, e.g. stainless steel and memory-shape alloys, such as nitinol, plastics, and even biodegradable polymer material.

Stents are inserted through a catheter and are then deployed remotely into their final shape at the target site. This deployment can be accomplished by radial pressure, as from distension of a balloon that is inside the stent, or by natural expansion of a shape-memory alloy that responds to elevated body temperature to relax into a distended, predetermined shape.

Stenting results in the largest lumen possible and expands the vessel to the greatest degree possible. However, the vessel may become partially or completely occluded in the region near or within the implanted device over time. This restenosis is a major problem in many therapies such as percutaneous coronary interventions because it causes repeated procedures and surgeries.

In-stent restenosis continues to be a significantly limiting factor in the intermediate and long term success of stent procedures. The etiology and biochemistry of this process are not entirely understood. At a minimum, restenosis must entail adhesion of myofibroblastic colloidal material, proteins, cells, and the like to the surface of the stent. Additionally, injury to the vessel endothelium during device delivery and deployment results in the exposure of subintimal collagen, lipids, and release of what is known as the von Willebrand factor. This produces platelet activation and adhesion, release of inflammatory factors, as well as migration and proliferation of smooth muscle cells and fibroblasts in the area of injury, and results in the formation of neointima, a composition of smooth muscle-like cells in a collagen matrix.

One approach to inhibiting restenosis is coating of the stent with an anti-inflammatory or antiproliferative pharmaceutical agent such as SIROLIMUS or PACLITAXEL. These agents interfere with the cell cycle, limit cell proliferation, and are thought to reduce restenosis. A problem with this approach is that the elution rate and duration of efficacy of the pharmaceutical agent is difficult to control, and the time scale for restenosis can span from weeks to years.

A second difficulty with coated stents generally, and drug eluting stents particularly, is that the coating material from which drug is eluted undergoes dramatic strain when the stent is expanded, as a result of a lack of control of the surface morphology and composition of the coatings during manufacture. Furthermore, to the extent that the coating material is brittle, it can fracture and delaminate during deployment. In vitro studies have shown that as much as 40% of the pharmaceutical coating is lost during stent deployment.

To further exacerbate the problems associated with stent deployment and restenosis, the stent metal structure itself can fracture upon deployment, or in use. Surface microfractures, are produced by current finishing techniques such as laser machining, electrodeposition, electropolishing, chemical etching, and the like. These microfractures can initiate brittle fracture of the stent both during deployment and when anatomical stresses are applied, resulting in device fragmentation and mechanical failure.

Another difficulty with prior art stents is that the morphology of surfaces that are presented to fluid flow have not, heretofore, been optimized or controlled. The present inventors have determined that the texture of the surface on the length scales appropriate to colloidal particles is crucial if one is to inhibit adhesion of these particles and the onset of restenosis.

Yet another difficulty with existing stents is that the surfaces are made with metals that form stable oxides. Stainless steels and titanium-nickel alloys are among the most widely used, and oxide at their surface engenders hydrogen bonding with colloidal particles present in blood, lymph, urine, bile and other bodily fluids, thereby initiating the formation of blockages within the stent. Hydrogen bonding results form the combined electrostatic, dipole, and covalent interactions between an electron deficient hydrogen atom bound, for example to oxygen, and an electron rich moiety such as oxygen, nitrogen, sulfur, or unsaturated carbon-carbon bonds.

### PRIOR ART

U.S. Pat. Nos. 5,746,691 , 6,086,455 and 6,537,202 to Frantzen disclose a method for polishing radially expandable surgical stents where fluid abrasive media flows over surfaces of the stent causing the surfaces of the stent to be polished and streamlined, which more effectively supports a body lumen without excessive thrombus, restenosis and other medical complications. An interior polishing fixture is provided which has cylindrical chambers adapted to receive a stent therein. Fluid abrasive media then flows into bores in the fixture leading to the cylindrical chambers and adjacent the inner diameter surfaces of the stent. The outer diameter surfaces of the stent are polished by placing the stent within an exterior polishing fixture. After polishing is completed, the stent is ready for implantation and radial expansion within a body lumen. The disclosures state that it has been found effective and preferable to have abrasive media particle sizes between 0.008 and 0.0003 inches (i.e., 203.2 and 7.62 μm). In addition, diamond particles could be used as the abrasive media particle (see column 13, line 25 to 34,). Frantzen recognizes that the surfaces forming the inner diameter of the stent are polished to a level of smoothness determined by the particle size of the abrasive media and the amount of time abrasive media flows past the surfaces of the stent (column 3, lines 53-60, of the '691 patent).

U.S. Pat. No. 5,788,558, to Klein discloses a method and apparatus for deburring and rounding edges and polishing surfaces of radially expansible lumenal prostheses, such as stents and grafts. A stent is mounted onto a polishing apparatus and a flowable abrasive slurry is extruded through the apparatus in abrading contact with inner and outer surfaces and circumferential openings in the stent. To polish the cut surfaces and edges surrounding the openings, the abrasive slurry is introduced into an inner lumen of the stent and extruded radially outward through the openings. The inner and outer wall surfaces are preferably pre-polished prior to cutting the slot pattern in the stent. The media is filled with an appropriate charge of abrasive grain, such as diamond. The abrasive particle size ranges from 0.005 mm to 1.5 mm, (5 μm to 1500 μm) see column 9, lines 5 to 18.

U.S. Pat. No. 5,207,706, to Menaker discloses implantable vascular prostheses, formed of synthetic, woven fibers coated with a thin layer of metallic gold sufficient to create a continuous coating over the surfaces of the fibers that come into contact with blood. The coating is applied by vapor deposition or sputtering to coat the fibers without blocking or bridging the interstices formed by the intersection of the fibers. The references shows that artificial expedients made from bio-compatible fluoropolymers (i.e., polytetrafluoroethylene) are conventional.

U.S. Pat. No. 5,824,056, to Rosenberg, discloses an implantable medical device formed from a drawn refractory metal and having an improved bio-compatible surface. The method by which the device is made includes coating a refractory metal article with platinum by a physical vapor deposition process and subjecting the coating article to drawing in a diamond die. The drawn article can be incorporated into an implantable medical device without removing the deposited metal.

U.S. Pat. No. 6,820,676, to Palmaz et al.; discloses an implantable endoluminal device which is fabricated from materials which present a blood or body fluid and tissue contact surface which has controlled heterogeneities in material constitution and which may include a synthetic or biologically active or inactive coating material such as a polymeric material (polytetrafluoroethylene). An endoluminal stent is disclosed made from a material (i.e., platinum, palladium, or gold) having substantially homogeneous surface properties in the stent material along the blood flow surface of the stent, specifically surface energy and electrostatic charge. The reference further discloses that irregular or unpredictable distribution of attachment sites that might occur as a result of various inclusions, with spacing equal or smaller to one whole cell length, is likely to determine alternating and favorable attachment conditions along the path of a migrating cell.

U.S. Published Patent Appl. No. 2005/0228490, Published Oct. 13, 2005, to Hezi-Yamit et al., discloses an implantable device having anti-restenotic coatings. Specifically, implantable devices having coatings of certain anti-proliferative agents (particularly BSM-181176). The medical device can be coated using any method known in the art including compounding the antiproliferative agent with a bio-compatible polymer prior to applying the coating. Additionally, medical devices having a coating comprising at least one anti-proliferative agent in combination with at least one additional therapeutic agent are also disclosed.

These references fail to teach or suggest utilization of a nano-abrasive finishing technique utilizing abrasive particles having dimensions between about 1 μm and 5 nm, which dimensions are commensurate with the dimensions, on a length scale, of colloidal particles found in bodily fluids, for controlling the surface finish.

The references further lack any suggestion of applying a thin coating of between 1 μm and 5 nm of a noble metal or hydrophobic coating to the surface of a nano-abrasively polished stent to preclude hydrogen bonding between the surface of the medical device and colloidal particles.

Although the prior art has disclosed noble metal or polymeric coating of implantable devices, such coatings have failed to result in substantial reduction of colloidal binding, in and of themselves. See for example Shabalovskaya et al, Institute for Physical Research and Technology, wherein gold coating of nitinol stents did not appreciably reduce the adsorption of proteins to the surfaces thereof.

Combining the two aspects of the present invention, that is, the step of polishing the surfaces of an implantable device so that they are featureless at length scales commensurate with the sizes of colloidal particles in biological fluids, and changing the surface chemistry to impede hydrogen bond formation between the devices and the colloidal particles, has not been disclosed by any prior art (i.e., between 1μm and 5 nm) coupled with treating the surface of the device with a thin coating of a biologically compatible material to inhibit oxidation and associated hydrogen bonding between a colloidal particle and the surface of the device, has not heretofore been disclosed by any of the prior art, and unexpectedly satisfies the long-felt need of producing an implantable device having reduced tendency toward restenosis and occlusion by adhesion of colloidal materials.

### SUMMARY OF THE INVENTION

The present invention provides a device and a method for reducing, and possibly eliminating, restenosis, the adhesion of colloidal particles from biological fluids onto the surfaces of implantable devices such as stents, prosthetic joints, catheters, and the like. This objective is accomplished by recognizing the fundamental coupling between the surface texture and composition, and the drag and adhesive forces acting on a colloidal particle. There are two components to the invention. First, at least one of the surfaces of the device, e.g. those that are exposed to fluid flow, are polished by an abrasive media, so that they are featureless on length scales that are commensurate with the sizes of colloidal particles that initiate restenosis. First, one or more surfaces of the device are polished with abrasive media so that they are featureless on the length scales that characterize colloidal particles that lead to deposits. Second, the surface is selectively treated with a thin coating that reduces, or preferably eliminates, hydrogen bonding with colloidal particles. In so far as polishing increases the practical fluid dynamical drag force and coating reduces the adhesive force, the combined impact of these steps reduces both the probability and duration with which colloidal particles that nucleate restenosis bind to the stent.

Summarizing, there are two forces that lead to binding of small colloidal particles on the surface of an implantable stent or the like implantable device: fluid dynamical drag and molecular adhesive forces. The present invention seeks to provide a mechanism to enhance fluid dynamical drag by polishing surfaces to prevent fluid stagnation on spatial scales that are commensurate with the size of the colloidal precursors to stenosis. The invention further provides a mechanism for reducing hydrogen bonding of the colloidal particles by producing an oxygen free surface in contact with the biological fluid.

Accordingly, it is an objective of the instant invention to provide a process for polishing the surface of an implantable device with a micro or nano-abrasive media to render the surface of the device featureless on length scales that are commensurate with the sizes of colloidal particles that initiate restenosis.

It is a further objective of the instant invention to provide a process for coating a micro or nano-abrasively polished implantable device with a coating effective for reducing, or possibly eliminating, hydrogen bonding with colloidal particles, on the surface of the polished implantable device.

It is yet a further objective to provide an implantable device and process for its manufacture which results in targeted reduction of structural micro-anomalies in such devices and targeted reduction or elimination of the propensity for occlusive deposits to form therein , whereby properties of selective tissue adherence are derived.

Other objects and advantages of this invention will become apparent from the following description, wherein are set forth, by way of illustration and example, certain embodiments of this invention.

### BRIEF DESCRIPTION OF THE DRAWINGS

FIG. 1. is a cross-sectional model of a 1 mm diameter blood vessel;

FIG. 2. is a graph that illustrates the velocity field for the model in FIG. 1; arrows depict the velocity direction and magnitude, with the first set of vectors between −4 and −3.6 mm scaled to 20 cm/s velocity;

FIG. 3 is a graph that illustrates a depression showing contours of horizontal velocity. This velocity is zero at the wall; contours are spaced at intervals of 2.5mm/s with a maximum of 2 cm/s;

FIG. 4A is a graph that illustrates the horizontal velocities in the vicinity of the indentation.

FIG. 4B is a chart that illustrates the x-velocity as a function of the vertical (y) coordinate along each of these vertical slices;

FIG. 5 is a chart that illustrates fluid flow in a channel with periodic protuberances;

FIG. 6 is a schematic drawing of a colloidal particle (602) adjacent to a vessel wall (603) that is being impinged upon by a flow of fluid (601);

FIG. 7A is a chart that illustrates horizontal velocity profiles inside and beyond the indentation.

FIG. 7B is a graph that illustrates the velocity profiles set forth in FIG. 7A.

### DETAILED DESCRIPTION OF THE INVENTION

The invention is applicable to any implantable device wherein it is desirable to provide surface modification to modify tissue adherence properties thereof. In a preferred, albeit non-limiting embodiment of the invention, an uncoated metal stent manufactured in accordance with known art is immersed in an aqueous colloidal suspension of abrasive particles, which particles may be defined as nano-abrasive particles, whose sizes are substantially less than 1 μm, preferably less than 100 nm, and ideally between 5 and 50 nm. Illustrative, albeit non-limiting examples of nano-abrasive particles are inorganic oxides such as Al₂O₃, SiO₂, CeO₂, and ZrO₂, and nano-diamonds, such as those available from NanoBlox, Inc., manufactured in accordance with U.S. Pat. Nos. 5,916,955 and 5,861,349, the contents of which are herein incorporated by reference. Illustrative, albeit non-limiting examples of micro-abrasive particles include inorganic oxides, crushed glass, glass beads, plastic media, silicon carbide, sodium bicarbonate, walnut shells, and the like, having particle sizes within the range of about 10 μm to 250 μm. A vibratory fluidized bed is formed wherein the suspension is excited by ultrasonic agitation at an amplitude and for a duration that is empirically determined to produce a particularly desired surface texture, e.g. a surface texture that is featureless on the spatial scales between 10 nm and 10 μm. In the case of a nano-abrasively polished stent, additional surface finishing can subsequently be accomplished by application of a thin film of less than 1 μm, preferably less than 100 nm, and ideally between 5 and 50 nm, of a material whose chemical composition lacks the capacity to form hydrogen bonds with biological colloids, illustrated by the noble metals Pd, Pt, Au, and organic polymers that lack accessible electronegative substituents such as N, O, and S provided that they maintain a smooth surface texture on the length scale of the colloidal particles. Alternatively, the polishing step may follow the coating step to produce a surface that is both featureless on the aforesaid length scales and unable to form hydrogen bonds.

The coating may be applied by electrolytic or electroless plating, vacuum sputtering, metalorganic chemical vapor deposition (MOCVD), plasma enhanced chemical vapor deposition, or other methods known to those practiced in the art of metal finishing.

One aspect of the preferred embodiment recognizes that thinner coatings are more likely to be plastically deformable (i.e. malleable) when the stent is deployed, so that the thinnest possible layer that is consistent with impeding hydrogen bonding is preferred.

Another aspect of the invention is that the coating is only critical on the surfaces that are exposed to fluid flow. Therefore a pharmaceutical agent may be applied to surfaces that are not impinged upon by fluid flow after the polishing and plating of the remaining surfaces. For example, a stent with cylindrical symmetry will, when deployed, have its outer surface in contact with the vessel endothelium. This surface may be coated with stenosis inhibiting drugs or the like, while the internal surfaces that contact the flowing blood are polished and finished according to the present invention.

It may be desirable to only target certain surfaces for texture modification, or to provide differentials in texture or surface characteristics. Such targeting will be effective in order to form areas of the device which have selective tissue adherence, or to impart selective structural properties to certain areas of the implant. It is within the purview of the instant invention to therefore utilize modifications to the sizes and types of micro and/or nano abrasive particles which are utilized, either singly or in a particularly desirable combination, in order to achieve the desired selectively targeted properties.

The method of the present invention may reduce the number and severity of microfractures on the device's surface. To the extent that brittle fracture is initiated by these surface defects the present invention reduces device failure under the stresses and strains that occur during deployment and under biomechanical processes.

In the preferred embodiment, the stent or other implantable device is polished in vitro prior to implantation.

Although application of the invention to vascular stents has been described in the preferred embodiment, the same principles apply to other implantable medical devices used in both the vascular and non-vascular systems, such as implantable artificial organs or parts thereof, e.g. artificial hearts, and heart valves, and implantable joint structures such as hip, knee, or shoulder joints, and implantable dental devices. These may further include metallic devices such as, Inferior Vena Cava Filters, Cardiac Pacemakers, artificial cardiac valves, artificial venous valves, vascular ports and the like. Additionally, the invention may be applied to non-metal medical implantable devices such as venous catheters, port catheters, biliary catheters, urinary catheters, drainage catheters and the like.

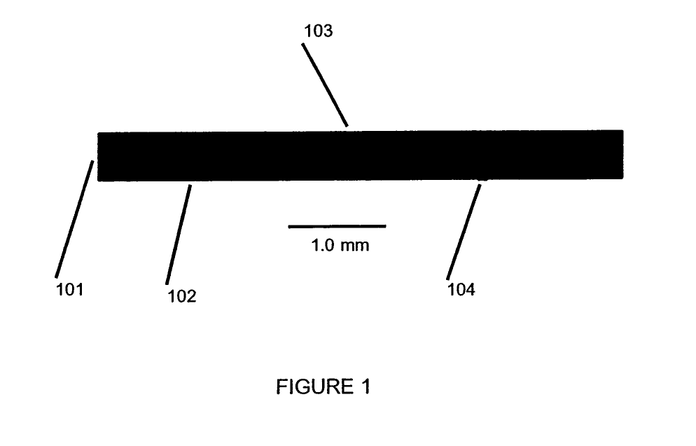

Now referring to FIG. 1, a model cross-section of a fluid vessel such as an artery, vein, bile duct, lymphatic vessel, renal duct, or the like is illustrated. Fluid enters at 101 and, in the model, experiences a slip boundary condition until it reaches the wall at 102, where the no-slip boundary condition on the Navier-Stokes fluid equations is applied. This generates velocity shear near the wall, with a parabolic velocity profile developing thereafter. The flow develops for 3 vessel diameters, where a surface depression 20 μm deep and 100 μm wide (104) is encountered. A symmetric boundary condition is applied at the centerline (103). The axis of symmetry is at the upper edge 103 of the drawing. A rectangular indentation whose size is commensurate with an epithelial cell (20 μm deep ×100 μm long) is included at 104.

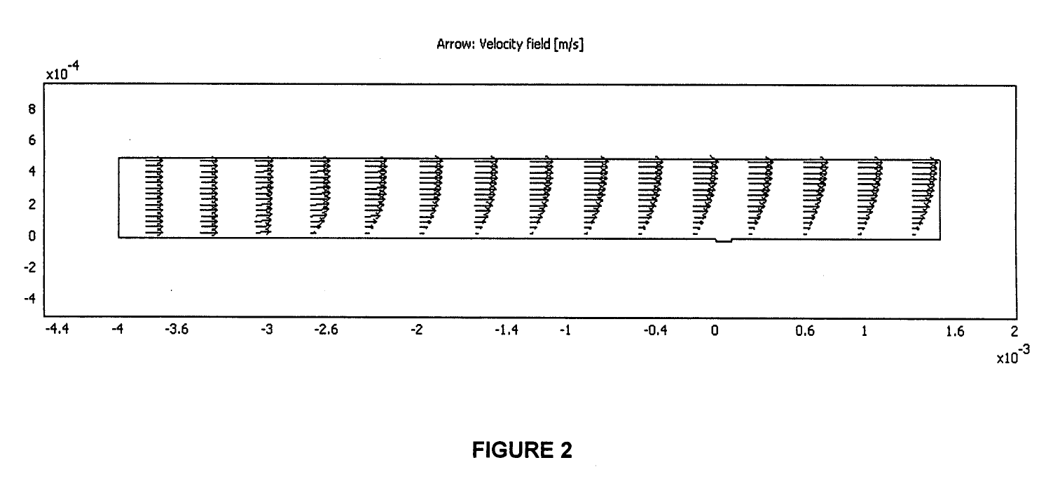

Solution of the Navier-Stokes fluid equations results in velocity profiles for the flow in the vessel as shown in FIG. 2. A non-limiting embodiment illustrates peak velocity at the centerline is 30 cm/s, with a standard Blausius profile to the smooth section of the wall.

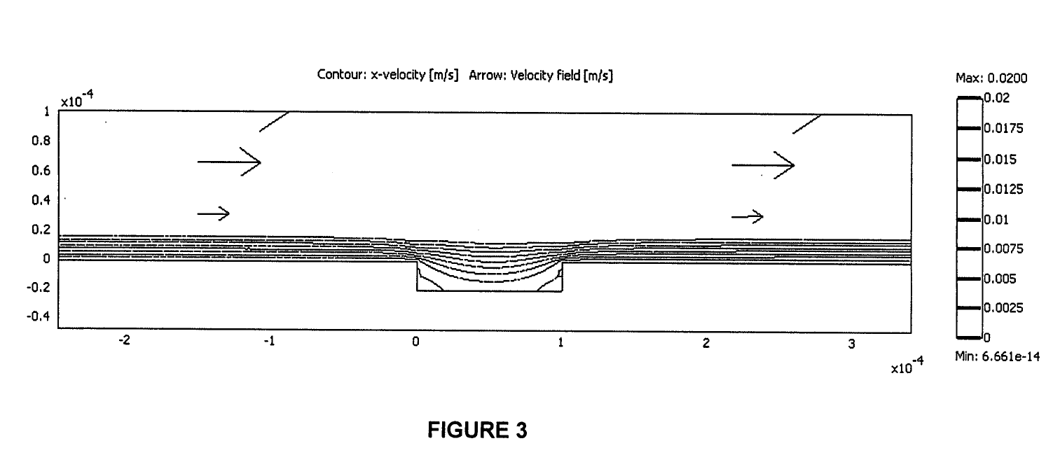

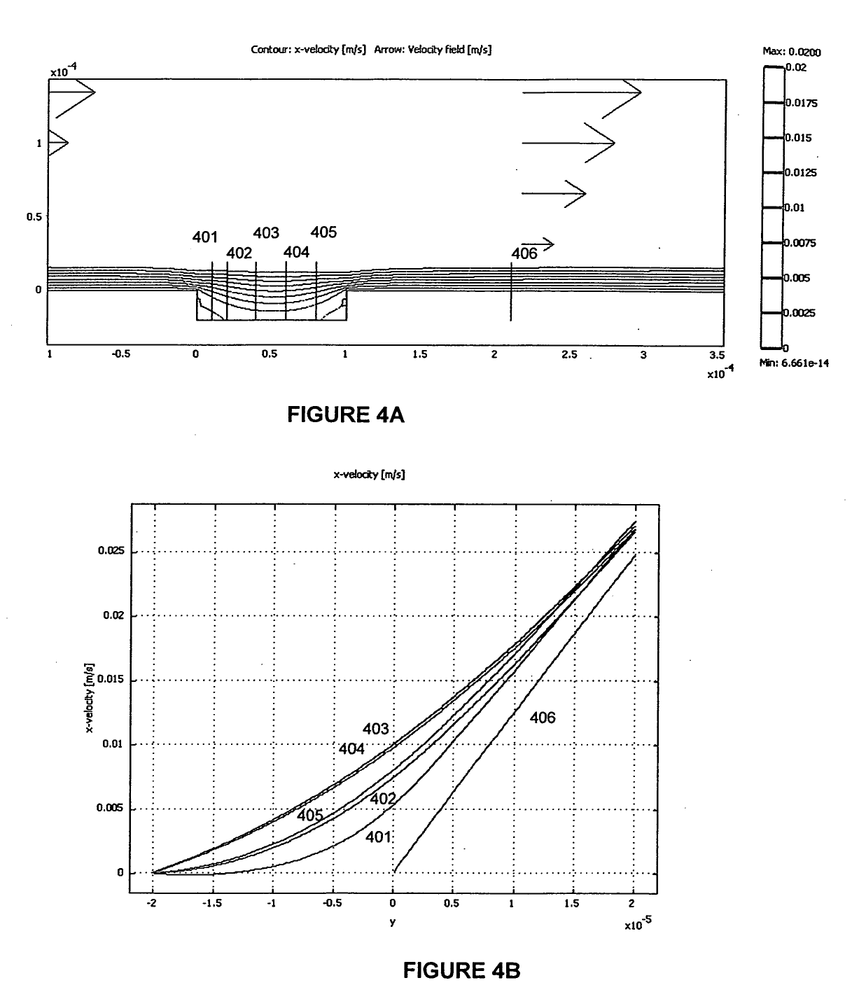

With reference to FIG. 3, an expanded view of the calculation showing contours of horizontal x-velocity in the vicinity of the indentation is shown. The dynamic pressure exerted on a particle suspended in the fluid is the product of the fluid density and the x-velocity. The horizontal force is the product of the pressure and the cross-sectional area of the particle. The importance of this force can be better understood with reference to FIGS. 4A & 4B.

FIGS. 4A and 4B show the horizontal or x-velocity both within and beyond the indentation. The locations of velocities within (401-405) and beyond (406) the depression are indicated in FIG. 4A. FIG. 4B displays the x-velocity as a function of the vertical (y) coordinate along each of these vertical slices. The average x-velocity within 20 μm of the wall outside of the indentation is 1.24 cm/s. The average x-velocities within 20 μm of the wall within the indentation at locations 401,402,403,404, and 405 are 0.12, 0.25, 0.42, 0.44, and 0.28 cm/s, respectively. In other words, the horizontal drag force on a particle within the indentation is reduced from what is experienced at the normal wall by a factor of 3 to 10 in this example.

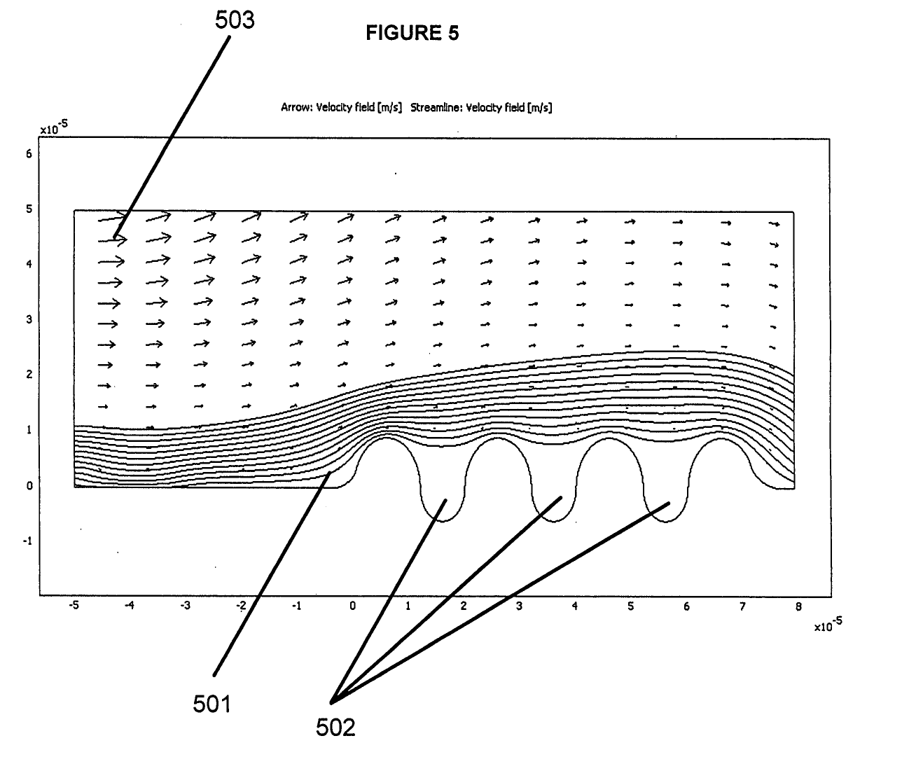

Now referring to FIG. 5, results from a second exemplar fluid dynamical calculation are displayed, where a periodic undulation in the surface reveals regions where the fluid velocity is diminished (502) and streamlines (501) reveal that corresponding drag forces are reduced. The protuberances have a modulation depth of 20 μm and a period of 20 μm. The length of the simulated region is 130 μm, and the region within 50 μm of the wall is displayed. Velocity vectors (503) and streamlines (501) illustrate the stagnation of flow inside the depressions (502). The length scale of the vector (503) corresponds to a velocity of 3 mm/s.

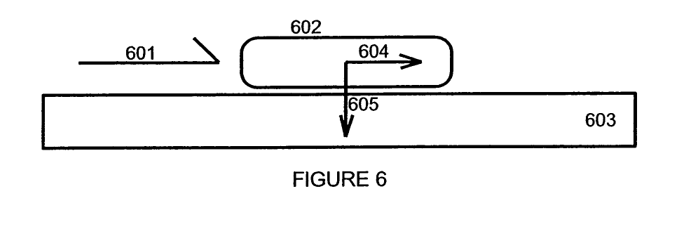

The second facet of the present invention can be understood with reference to FIG. 6. A colloidal particle (602) flowing in the biological fluid is shown schematically adjacent to a boundary of the implantable device (603). As described previously, fluid dynamical drag caused by momentum transfer from the moving fluid (601) results in a force whose direction and magnitude are indicated by the vector (604). At the same time, chemical and physical interactions between the particle (602) and surface (603) from electrostatic attraction, hydrogen bonding, dispersion (or van der Waals) interactions, or other forms of chemical binding lead to an adhesive interaction indicated schematically by the force vector (605). If the shear force (604) is much larger than the adhesive force then the laws of mechanics will preclude permanent binding of the particle to the wall. Conversely, if the strength of the adhesive force (605) is adequate to prevent shear in the presence of drag force (601) then the particle will remain adhered to the surface.

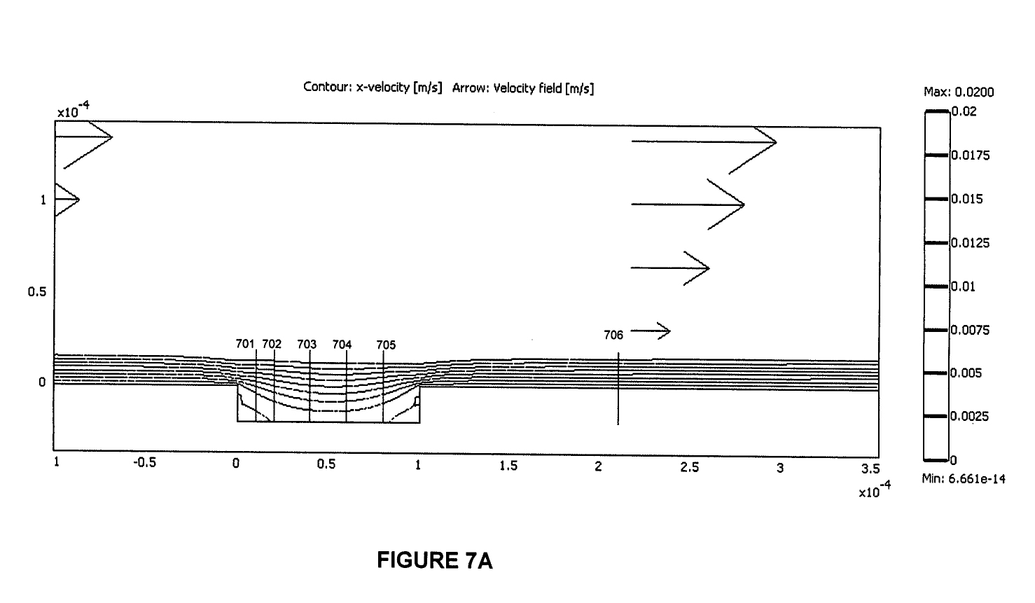

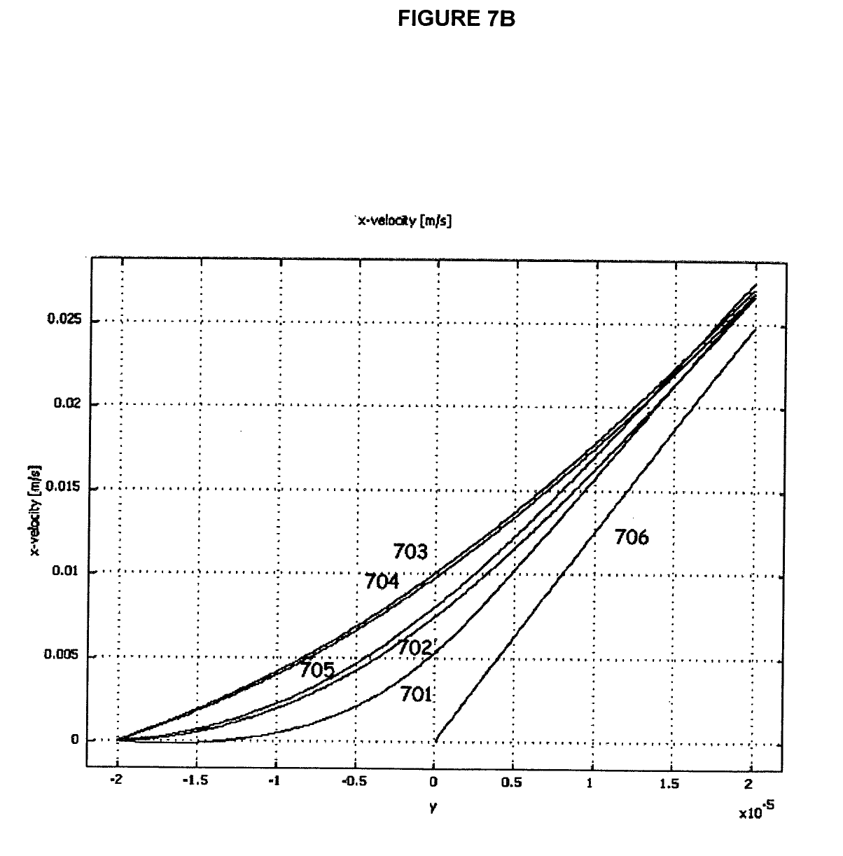

Now referring to FIGS. 7A & 7B, horizontal velocity profiles inside (701-705) and beyond (706) the indentation is shown. FIG. 7B displays the x-velocity as a function of the vertical (y) coordinate along each of these vertical slices. The location of the positions corresponding to velocities in FIG. 7B are labeled in FIG. 7A.

Prior art stents are made from alloys that include oxidized states of metals such as iron, titanium, and the like. Oxides at the surface of these stents are able to form hydrogen bonds with colloidal particles that have hydroxyl functionalities on their surface, a result which is generally true of these biomolecules to an extent that depends in detail on their chemical composition and conformation in the suspension. The second aspect of the present invention recognizes that certain metals such as platinum, palladium, and gold do not form stable oxides. Therefore coating of the stent with a thin layer of one of these metals by electrodeposition, sputtering, metal-organic chemical vapor deposition, plasma spraying, or the like will prevent hydrogen bonding of colloidal particles, thereby reducing the magnitude of the adhesive force (605). An alternative embodiment of the invention would provide a hydrophobic coating such as a flexible fluoropolymer to preclude hydrogen bonding by colloidal particles.

Polishing of the implant surface, particularly the part of the surface that is in contact with flowing biological fluids imparts properties of selective tissue adherence, and can be accomplished by a variety of means familiar to those practiced in the art of surface finishing. In a preferred embodiment, a stainless steel or nitinol stent is immersed in and subjected to, a moving colloidal suspension containing micro and/or nano-abrasive particles. These particles are chosen to have a size, shape, and hardness effective to produce a surface finish that is smooth at the spatial scale corresponding to the size of colloidal particles in the biological fluid. For example, endothelial cells which may adhere to a stent and lead to occlusion are typically disk shaped with lateral dimensions of 10 μm and thickness of 1-2 μm. Leukocytes, neutrophils, and granulocytes have diameters in the 10-15 μm range.

TABLE I
Typical dimensions of colloidal particles in vivo

| | |
|---|---|
| Endothelial cell | 1 μm thick, 10 μm diameter |
| Basophil | 5–7 μm |
| Monocyte | 12–20 μm |
| Lymphocyte | 5–12 μm |
| Red Blood Cells | 2 μm thick, 7 μm diameter |
| Fibrinogen/Fibrin | 90 nm diameter, μm in length |
| Factor VIII (clotting protein) | 4 × 6 nm to 8 × 12 nm |
| Low density Lipoprotein | 10–20 nm diameter |
| Platelets | 1–4 μm diameter |

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and drawings/figures. One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A device implanted in a flowing biological fluid characterized by the drag force on colloidal particles in the fluid exceeds the adhesive force between the particles and the device.

2. An implantable device having a surface which is essentially featureless on a spatial scale commensurate of the colloidal particles flowing within or around the device.

3. The implantable device according to claim 2 with a surface which is featureless and which includes a surface coating which renders said surface essentially inert to hydrogen bonding of flowing colloidal material.

4. Method for producing a device having a surface which is essentially featureless on a spatial scale conimensurate of the colloidal particles flowing within or around the device comprising:
immersing said device in a fluid with abrasive particles whose size is chosen to produce a surface finish on a length scale to inhibit adhesion of circulating colloidal particles.

5. A method to produce a no-hydrogen bond surface comprising application to said surface of a noble metal deposition and O₂ free polymer.

6. A method to produce a non-adhesive smooth surface comprising smoothing the surface to a length scale commensurate with the circulating colloidal particles and subsequently applying a coating to produce a hydrogen bonding free surface.

7. A method to produce a non-adhesive smooth surface comprising applying a surface coating to produce a hydrogen bonding free surface and subsequently polishing the surface to a length scale effective to inhibit adhesion of circulating colloidal particles.

8. The device of any one of claim 1, or claim 2 or claim 3 selected from a vascular stent, filter or other implantable metal endovascular device.

9. The device of any one of claims 1 or 2 or 3 wherein said coating is selected from a group of nobel metals such as gold, palladium and platinum, or an O₂ polymer.
